(19) United States
(12) Patent Application Publication
(10) Pub. No.: US 2025/0232770 A1
(43) Pub. Date: Jul. 17, 2025

(54) ELECTRONIC DEVICE AND METHOD FOR PROCESSING USER UTTERANCE IN ELECTRONIC DEVICE

(71) Applicant: SAMSUNG ELECTRONICS CO., LTD., Suwon-si (KR)

(72) Inventors: Hyunwoong LIM, Suwon-si (KR); Gajin SONG, Suwon-si (KR); Kyungtae KIM, Suwon-si (KR)

(21) Appl. No.: 19/171,150

(22) Filed: Apr. 4, 2025

Related U.S. Application Data
(63) Continuation of application No. PCT/KR2023/015235, filed on Oct. 4, 2023.

(30) Foreign Application Priority Data
Oct. 6, 2022 (KR) 10-2022-0127490
Oct. 20, 2022 (KR) 10-2022-0135512

(51) Int. Cl. G10L 15/22; G06F 3/16

(52) U.S. Cl. CPC G10L 15/22; G06F 3/167; G10L 2015/225

(57) ABSTRACT

According to an embodiment, an electronic device, in case that a user utterance related to a date and time acquired through the input module is identified as a user utterance for performing a function, based on a date and time when the user utterance is acquired, identifies whether the date and time detected in the user utterance are interpreted as a plurality of dates and times. According to an embodiment, the electronic device, in case that the date and time detected in the user utterance are interpreted as the plurality of dates and times, changes the date detected in the user utterance if a first condition for changing the date is satisfied, and changes the time detected in the user utterance if user's activity record information satisfies a second condition. According to an embodiment, the electronic device recommends performing a function with the changed date or time.

### CROSS-REFERENCE

This application claims priority to PCT Application No. PCT/KR2023/015235, filed on Oct. 4, 2023, Korean Patent Application No. 10-2022-0127490, filed on Oct. 6, 2022, and Korean Patent Application No. 10-2022-0135512, filed on Oct. 20, 2022, and all the benefits accruing therefrom under 35 U.S.C. § 119, the contents of which in their entirety are herein incorporated by reference.

### BACKGROUND

### Technical Field

The disclosure relates to an electronic device capable of detecting a user intention to change a date and a time included in a user utterance to and perform a function and a method of processing the user utterance in the electronic device.

### Background Art

Portable digital communication devices have become useful to many people living in modern times. Consumers want to receive various desired high-quality services anywhere and at any time through portable digital communication devices.

A voice recognition service is a service that provides consumers with various content services in response to a user voice received based on a voice recognition interface implemented in portable digital communication devices. In order to provide the voice recognition service, the portable digital communication devices realize technologies of recognizing and analyzing human languages (for example, automatic voice recognition, natural language understanding, natural language creation, machine translation, dialog system, question and answer, and voice recognition/synthesis).

In order to provide a high-quality voice recognition service to consumers, it is desired to implement a technology for accurately identifying a user intent and a technology for providing a suitable content service corresponding to the identified user intention, based on a user voice.

### SUMMARY

An electronic device according to an embodiment may include an input module, an output module, a processor, and a memory.

When a user utterance related to a date and time acquired through an input module is identified as a user utterance for performing a function, the processor according to an embodiment may identify whether the date and time detected in the user utterance are interpreted as a plurality of dates and times, based on a date and time when the user utterance is obtained.

When the date and time detected in the user utterance are interpreted as the plurality of dates and times, the processor according to an embodiment may change the date detected in the user utterance if the date and time when the user utterance is obtained satisfy a first condition for changing the date, and change the time detected in the user utterance if user's activity record information satisfies a second condition for changing the time.

The processor according to an embodiment may recommend performance of the function on the changed date or time through the output module.

A method of processing a user utterance by an electronic device according to an embodiment may include, when a user utterance related to a date and time acquired through an input module is identified as a user utterance for performing a function, identifying whether the date and time detected in the user utterance are interpreted as a plurality of dates and times, based on a date and time when the user utterance is obtained.

The method according to an embodiment may include, when the date and time detected in the user utterance are interpreted as the plurality of dates and times, changing the date detected in the user utterance if the date and time when the user utterance is obtained satisfy a first condition for changing the date, and changing the time detected in the user utterance if user's activity record information satisfies a second condition for changing the time.

The method according to an embodiment may include recommending performance of the function on the changed date or time through an output module.

### BRIEF DESCRIPTION OF DRAWINGS

FIG. 1 is a block diagram illustrating an electronic device within a network environment according to an embodiment.

FIG. 2 is a block diagram illustrating an integrated intelligence system according to an embodiment.

FIG. 3 is a diagram illustrating the form in which relation information between concepts and actions is stored in a database according to an embodiment.

FIG. 4 is a diagram illustrating a user terminal which displays a screen for processing a voice input received through an intelligent app according to an embodiment.

FIG. 5 is a block diagram of the electronic device according to an embodiment.

FIGS. 6A to 6B are diagrams illustrating an operation in which the electronic device sets a sleep time according to an embodiment.

FIGS. 7A to 7B are diagrams illustrating an operation in which the electronic device activates a function of changing a date and time according to an embodiment.

FIG. 8 is a diagram illustrating an operation in which the electronic device processes a user utterance according to an embodiment.

FIG. 9 is a diagram illustrating an operation in which the electronic device processes a user utterance according to an embodiment.

FIGS. 10A to 10B are diagrams illustrating an operation in which the electronic device processes a user utterance according to an embodiment.

FIG. 11 is a flowchart illustrating an operation in which a wearable electronic device processes a user utterance according to an embodiment.

FIGS. 12A, 12B, and 12C are flowcharts illustrating an operation in which the wearable electronic device processes a user utterance according to an embodiment.

### MODE FOR CARRYING OUT THE INVENTION

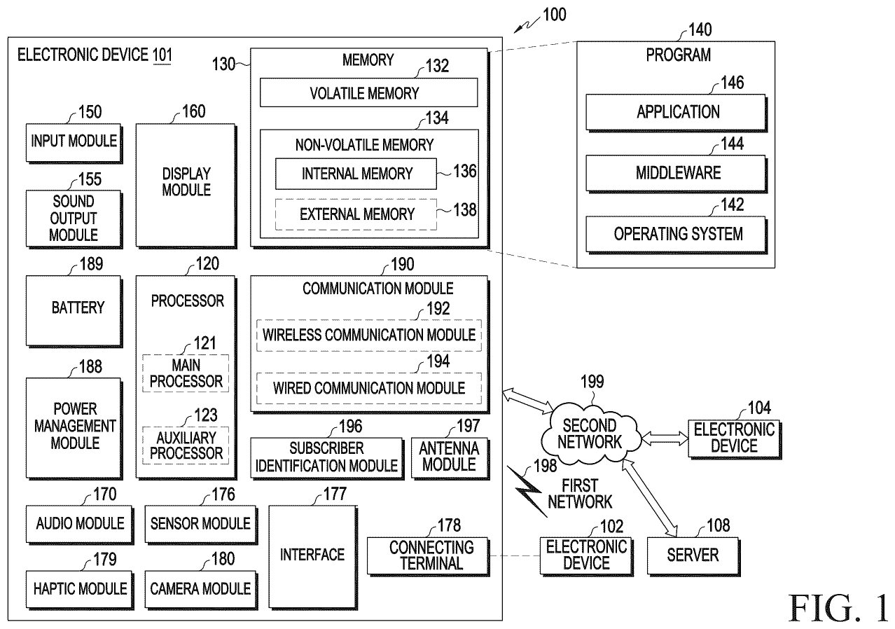

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™ wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

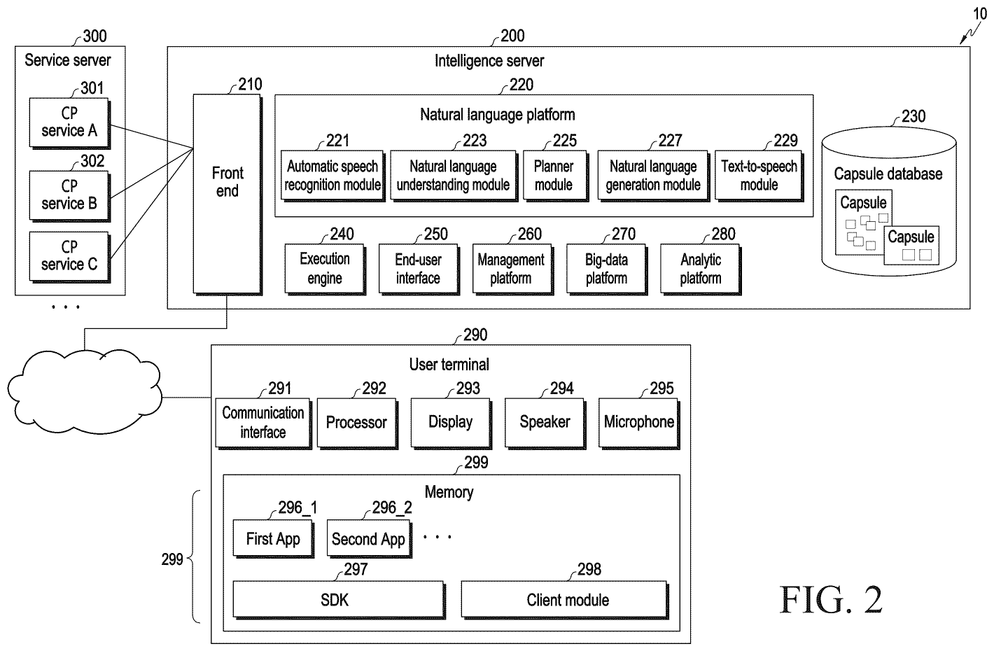

FIG. 2 is a block diagram illustrating an integrated intelligence system according to an embodiment.

Referring to FIG. 2, the integrated intelligence system 10 according to an embodiment may include a user terminal 290, an intelligent server 200, and a service server 300.

According to an embodiment, the user terminal 290 may be a terminal device (or electronic device) capable of connecting to the Internet, for example, a mobile phone, a smartphone, a personal digital assistant (PDA), a notebook computer, a TV, a white home appliance, an electronic device, an HMD, or a smart speaker.

According to the illustrated embodiment, the user terminal 290 may include a communication interface 291, a microphone 295, a speaker 294, a display 293, memory 299, or a processor 292. The listed elements may be operatively or electrically connected to each other.

According to an embodiment, the communication interface 291 may be connected to an external device and configured to transmit and receive data. The microphone 295 according to an embodiment may receive a sound (for example, user utterance) and convert the same into an electrical signal. The speaker 294 according to an embodiment may output an electrical signal in the form of a sound (for example, voice). The display 293 according to an embodiment may be configured to display an image or a video. The display 293 according to an embodiment may display a graphic user interface (GUI) of an executed app (or application program).

According to an embodiment, the memory 299 may store a client module 298, a software development kit (SDK) 297, and a plurality of apps 296 (e.g., applications). The client module 298 and the SDK 297 may configure a framework (or a solution program) for performing a universal function. Further, the client module 298 or the SDK 297 may configure a framework for processing a voice input.

According to an embodiment, the plurality of apps 296 stored in the memory 299 may be programs for performing a predetermined function. According to an embodiment, the plurality of apps 296 may include a first app 2961 and a second app 296_2. According to an embodiment, each of the plurality of apps 296 may include a plurality of operations for performing predetermined functions. For example, the apps may include an alarm app, a message app, and/or a schedule app. According to an embodiment, the plurality of apps 296 may be executed by the processor 292 so as to sequentially perform at least some of the plurality of operations.

According to an embodiment, the processor 292 may control the overall operation of the user terminal 290. For example, the processor 292 may be electrically connected to the communication interface 291, the microphone 295, the speaker 294, and the display 293 to perform predetermined operations.

According to an embodiment, the processor 292 may perform a predetermined function by executing a program stored in the memory 299. For example, the processor 292 may perform the following operation for processing a voice input by executing at least one of the client module 298 or the SDK 297. The processor 292 may control, for example, the operation of the plurality of apps 296 through the SDK 297. The following operation which is described as the operation of the client module 298 or the SDK 297 may be the operation by execution of the processor 292.

According to an embodiment, the client module 298 may receive a voice input. For example, the client module 298 may receive a voice signal corresponding to a user utterance detected through the microphone 295. The client module 298 may transmit the received voice input to the intelligent server 200. The client module 298 may transmit state information of the user terminal 290 along with the received voice input to the intelligent server 200. The status information may be, for example, execution state information of the app.

According to an embodiment, the client module 298 may receive the result corresponding to the received voice input. For example, when the intelligent server 200 obtains the result corresponding to the received voice input, the client module 298 may receive the result corresponding to the received voice input. The client module 298 may display the received result on the display 293.

According to an embodiment, the client module 298 may receive a plan corresponding to the received voice input. The client module 298 may display the result obtained by performing the plurality of operations of the app on the display 293 according to the plan. The client module 298 may sequentially display, for example, the execution result of the plurality of operations on display 293. According to one or more embodiments, the user terminal 290 may display, for another example, only some results (for example, the result of the last operation) of executing the plurality of operations on display 293.

According to an embodiment, the client module 298 may receive a request for acquiring information for obtaining the result corresponding to the voice input from the intelligent server 200. According to an embodiment, the client module 298 may transmit the acquired information to the intelligent server 200 in response to the request.

According to an embodiment, the client module 298 may transmit result information acquired by performing the plurality of operations to the intelligent server 200 according to the plan. The intelligent server 200 may identify that the received voice input is correctly processed using the result information.

According to an embodiment, the client module 298 may include a voice recognition module. According to an embodiment, the client module 298 may recognize a voice input for performing a limited function through the voice recognition module. For example, the client module 298 may execute an intelligent app for processing a voice input to perform an organic operation through a predetermined input (for example, “wake up!”).

According to an embodiment, the intelligent server 200 according to an embodiment may receive information related to a user voice input from the user terminal 290 through a communication network. According to an embodiment, the intelligent server 200 may change data related to the received voice input into text data. According to an embodiment, the intelligent server 200 may generate a plan for performing a task corresponding to the user voice input, based on the text data.

According to an embodiment, the plan may be generated by an artificial intelligence (AI) system. The integrated intelligence system 10 may be a rule-based system, a neural network-based system (for example, a feedforward neural network (FNN)), or a recurrent neural network (RNN)). Alternatively, the integrated intelligence system 10 may be a combination thereof or an intelligent system different therefrom. According to an embodiment, the plan may be selected from a combination of predefined plans or may be generated in real time in response to a user request. For example, the artificial intelligent system may select at least one plan from among the plurality of predefined planes.

According to an embodiment, the intelligent server 200 may transmit the result of the generated plan to the user terminal 290 or transmit the generated plan to the user terminal 290. According to this configuration, the user terminal 290 may display a result according to the plan on the display 293. According to an embodiment, the user terminal 290 may display the result of performing the operation according to the plan on the display 293.

According to an embodiment, the intelligent server 200 may include a front end 210, a natural language platform 220, a capsule database (DB) 230, an execution engine 240, an end user interface 250, a management platform 260, a big data platform 270, or an analytic platform 280.

According to an embodiment, the front end 210 receive the voice input received from the user terminal 290. The front end 210 may transmit a response corresponding to the voice input.

According to an embodiment, the natural language platform 220 may include an automatic speech recognition (ASR) module 221, a natural language understanding (NLU) module 223, a planner module 225, natural language generator (NLG) module 227, or a text to speech (TTS) module 229.

According to an embodiment, the automatic speech recognition module 221 may convert the voice input received from the user terminal 290 into text data. The natural language understanding module 223 according to an embodiment may detect a user's intention, based on the text data of the voice input. For example, the natural language understanding module 223 may detect a user's intention by performing syntactic analysis or semantic analysis. The natural language understanding module 223 according to an embodiment may detect a meaning of a word extracted from the voice input by using a linguistic characteristic of a morpheme or a phrase (for example, grammatical element) and match the detected meaning of the word and the intention so as to determine the user's intention.

According to an embodiment, the planner module 225 may generate a plan by using the intention determined by the natural language understanding module 223 and a parameter. According to an embodiment, the planner module 225 may determine a plurality of domains for performing a task, based on the determined intention. The planner module 225 may determine a plurality of operations included in the plurality of domains determined based on the intention. According to an embodiment, the planner module 225 may determine a parameter for performing the plurality of determined operations or a result value output by the execution of the plurality of operations. The parameter and the result value may be defined by a concept of a predetermined type (or class). According thereto, the plan may include a plurality of actions determined by the user's intention and a plurality of concepts. The planner module 225 may operatively (or hierarchically) determine the relationship between the plurality of actions and the plurality of concepts. For example, the planner module 225 may determine the execution order of the plurality of operations determined based on the user's intention, based on the plurality of concepts. In other words, the planner module 225 may determine the execution order of the plurality of operations, based on the parameter for performing the plurality of operations and the result output by the execution of the plurality of operations. Accordingly, the planner module 225 may generate a plan including information on the relationship (for example, ontology) between the plurality of actions and the plurality of concepts. The planner module 225 may generate a plan, based on information stored in the capsule database 230 corresponding to a set of relationships between concepts and operations.

According to an embodiment, the natural language generator module 227 may change predetermined information in the form of text. The information converted into the form of text may be the form of a natural language speech. The text to speech module 229 according to an embodiment may convert information in the form of text into information in the form of voice.

According to an embodiment, some or all of the functions of the natural language platform 220 may be performed by the user terminal 290.

The capsule database 230 may store information on the relationship between a plurality of concepts and operations corresponding to a plurality of domains. The capsule according to an embodiment may include a plurality of action objects (or action information) and concept objects (or concept information) included in the plan. According to an embodiment, the capsule database 230 may store a plurality of capsules in the form of a concept action network (CAN). According to an embodiment, the plurality of capsules may be stored in a function registry included in the capsule database 230.

The capsule database 230 may include a strategy registry storing strategy information used when a plan corresponding to a voice input is determined. When there are a plurality of plans corresponding to the voice input, the strategy information may include reference information for determining one plan. According to an embodiment, the capsule database 230 may include a follow up registry storing information on the following action for suggesting the following action to the user in a predetermined situation. The following action may include, for example, the following speech. According to an embodiment, the capsule database 230 may include a layout registry storing layout information of the information output through the user terminal 290. According to an embodiment, the capsule database 230 may include a vocabulary registry storing vocabulary information included in the capsule information. According to an embodiment, the capsule database 230 may include a dialogue registry storing information on dialogue (or interaction) with the user. The capsule database 230 may update the stored object through a developer tool. The developer tool may include a function editor for updating, for example, the action object or the concept object. The developer tool may include a vocabulary editor for updating a vocabulary. The developer tool may include a strategy editor for generating and registering a strategy to determine a plan. The developer tool may include a dialogue editor that generates dialogue with the user. The developer tool may include a follow up editor capable of activating a follow up goal and editing a following speech that provides a hint. The follow up goal may be determined based on the currently set goal, a user's preference, or an environment condition. In an embodiment, the capsule database 230 can be implemented within the user terminal 290.

According to an embodiment, the execution engine 240 may obtain the result by using the generated plan. The end user interface 250 may transmit the obtained result to the user terminal 290. Accordingly, the user terminal 290 may receive the result and provide the received result to the user. According to an embodiment, the management platform 260 may manage information used by the intelligent server 200. According to an embodiment, the big data platform 270 may collect user data. According to an embodiment, the analytic platform 280 may manage quality of service (QoS) of the intelligent server 200. For example, the analytic platform 280 may manage elements and a processing speed (or efficiency) of the intelligent server 200.

According to an embodiment, the service server 300 may provide a predetermined service (for example, food order or hotel reservation) to the user terminal 290. According to an embodiment, the service server 300 may be a server operated by a third party. The service server 300 according to an embodiment may provide information for generating a plan corresponding to the received voice input to the intelligent server 200. The provided information may be stored in the capsule database 230. Further, the service server 300 may provide result information of the plan to the intelligent server 200.

In the integrated intelligence system 10, the user terminal 290 may provide various intelligent services to the user in response to a user input. The user input may include, for example, an input through a physical button, a touch input, or a voice input.

In an embodiment, the user terminal 290 may provide a voice recognition service through an intelligent app (or a voice recognition app) stored in the user terminal 290. In this case, for example, the user terminal 290 may recognize a user utterance or a voice input received through the microphone and provide a service corresponding to the recognized voice input to the user.

In an embodiment, the user terminal 290 may perform a designated action alone or together with the intelligent server 200 and/or the service server 300, based on the received voice input. For example, the user terminal 290 may execute an app corresponding to the received voice input and perform a predetermined action through the executed app.

According to an embodiment, when the user terminal 290 provides the service together with the intelligent server 200 and/or the service server 300, the user terminal 290 may detect a user utterance through the microphone 295 and generate a signal (or voice data) corresponding to the detected user utterance. The user terminal 290 may transmit the voice data to the intelligent server 200 by using the communication interface 291.

According to an embodiment, the intelligent server 200 may generate a plan for performing a task corresponding to the voice input or the result of the action according to the plan in response to the voice input received from the user terminal 290. The plan may include, for example, a plurality of actions for performing a task corresponding to the voice input of the user and a plurality of concepts related to the plurality of actions. The concepts may be parameters input by execution of the plurality of actions or may be defined for result values output by the execution of the plurality of actions. The plan may include the relationship between the plurality of actions and the plurality of concepts.

According to an embodiment, the user terminal 290 may receive the response through the communication interface 291. The user terminal 290 may output a voice signal generated within the user terminal 290 to the outside through the speaker 294 or output an image generated within the user terminal 290 to the outside through the display 293.

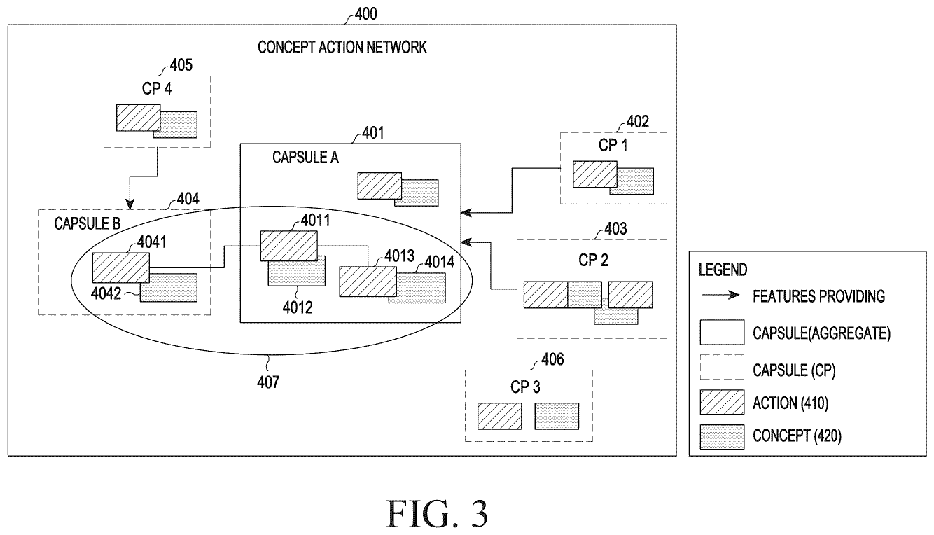

FIG. 3 is a diagram illustrating the form in which relation information between concepts and actions is stored in a database according to various embodiments.

A capsule database (for example, the capsule database 230) of the intelligent server 200 may store capsules in the form of a concept action network (CAN) 400. The capsule database may store an operation for processing a task corresponding to a user voice input and a parameter for the operation in the form of a concept action network (CAN) 400.

The capsule database may store a plurality of capsules (capsule A 401 and capsule B 404) corresponding to a plurality of domains (for example, applications). According to an embodiment, one capsule (for example, capsule A 401) may correspond to one domain (for example, location (geo) or application). In addition, one capsule may correspond to at least one service provider (for example, CP 1 402 or CP 2 403) for performing a function for a domain related to the capsule. According to an embodiment, one capsule may include one or more operations 410 for performing a predetermined function and one or more concepts 420.

The natural language platform 220 may generate a plan for performing a task corresponding to the received voice input by using the capsules stored in the capsule database. For example, the planner module 225 of the natural language platform may generate a plan by using capsules stored in the capsule database. For example, a plan 407 may be generated using actions 4011 and 4013 and concepts 4012 and 4014 of the capsule A 410 and an action 4041 and a concept 4042 of the capsule B 404.

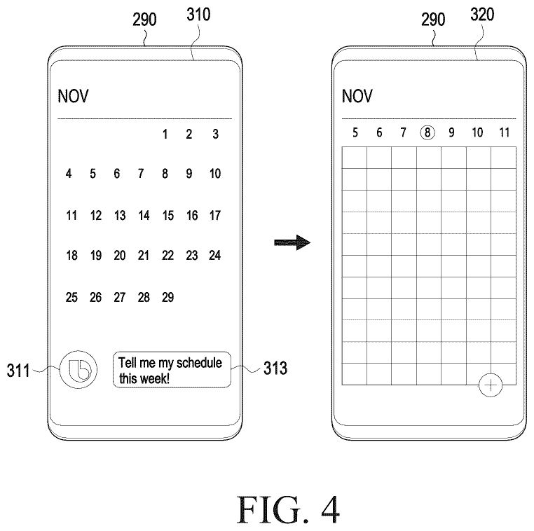

FIG. 4 is a diagram illustrating a screen in which a user terminal processes a voice input received through an intelligent app according to various embodiments.

The user terminal 290 may execute an intelligent app in order to process a user input through the intelligent server 200.

According to an embodiment, on a screen 310, when recognizing a designated voice input (for example, wakeup!) or receives an input through a hardware key (for example, a dedicated hardware key), the user terminal 290 may execute an intelligent app for processing the voice input. The user terminal 290 may execute the intelligent app in the state in which, for example, a schedule app is executed. According to an embodiment, the user terminal 290 may display an object 311 (for example, an icon) corresponding to the intelligent app on the display 293. According to an embodiment, the user terminal 290 may receive a voice input by a user utterance. For example, the user terminal 290 may receive a voice input of “Tell me my schedule this week”. According to an embodiment, the user terminal 290 may display a user interface (UI) 313 (for example, an input window) of the intelligent app displaying text data of the received voice input on the display.

According to an embodiment, on a screen 320, the user terminal 290 may display the result corresponding to the received voice input on the display. For example, the user terminal 290 may receive a plan corresponding to the received user input and display the “my schedule this week” on the display according to the plan.

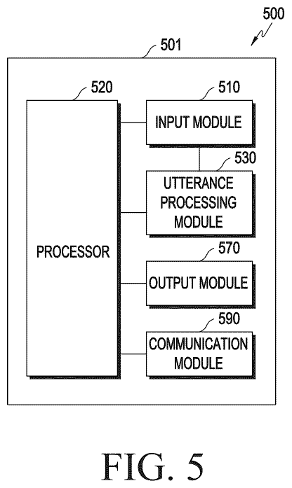

FIG. 5 is a block diagram 500 of an electronic device according to an embodiment.

Referring to FIG. 5, according to an embodiment, an electronic device 501 (for example, the electronic device 101 of FIG. 1 and/or the user terminal 290 of FIG. 2) may include a processor 520, an input module 510, an utterance processing module 530, an output module 570, and a communication module 590.

At least some of the components of the electronic device 501 shown in FIG. 5 may be the same as or similar to the components of the electronic device 101 of FIG. 1 and/or the components of the user terminal 290 of FIG. 2, and hereinafter, a redundant description will be omitted.

According to an embodiment, the processor 520 may be implemented substantially the same as or similar to the processor 120 of FIG. 1 and/or the processor 292 of FIG. 2.

According to an embodiment, when the user utterance related to the date and time obtained through the input module 510 is identified as the user utterance for function execution, the processor 520 may identify whether the date and/or time detected in the user utterance is interpreted as a plurality of dates and/or times, based on the date and time when the user utterance was obtained.

According to an embodiment, when receiving a user utterance through the input module 510, the processor 520 may identify the user's sleep time, based on the user's activity record information and activate a function of changing the date and time when the difference between an end time of sleep and a first reference time is smaller than the difference between a start time of sleep and the first reference time. According to an embodiment, the processor 520 may activate the function of changing the date and time when the condition of <Equation 1> below is satisfied, and deactivate the function of changing the date and time when the condition of <Equation 1> below is not satisfied.

min(sleep start time,24th−sleep start time)<min(sleep end time,24th−sleep end time) <Equation 1>

For example, when the sleep start time is 3:00 a.m. and the sleep end time is 10:00 a.m., the processor 520 may activate the function of changing the date and time by satisfying the condition from “min(3, 24^{th}−3=21)<min(10, 24^{th} −10=14)” to 3<10.

According to an embodiment, the processor 520 may activate the function of changing the date and time when the sleep end time is closer to midnight than the sleep start time through <Equation 1>.

According to an embodiment, when function of changing the date and time is activated, the processor 520 may identify whether the date and time detected in the user utterance are interpreted as a plurality of dates and times, based on the time when the user utterance is obtained.

According to an embodiment, the processor 520 may determine that the date and/or time is interpreted as a plurality of dates and/or times when the date detected in the user utterance is a non-explicit date that is not specified as a number, and the time detected in the user utterance is a non-explicit time that is not specified as a morning or afternoon.

The date detected in the user utterance that is the non-explicit date that is not designated as a number may include, for example, tomorrow, next week, last week, this year and/or next year.

According to an embodiment, when the date and time detected in the user utterance are interpreted as a plurality of dates and times, the processor 520 may identify whether the date and time when the user utterance is obtained satisfy a first condition for changing the date in the state where the date and time when the user utterance is obtained have passed the first reference time.

According to an embodiment, when the date and time when the user utterance is obtained satisfy “the first reference time <=the date and time when the user utterance is obtained <=a second reference time,” the processor 520 may identify that the date and time when the user utterance is obtained satisfy the first condition for changing the date.

According to this configuration, the processor 520 may configure the first reference time as a default time (for example, midnight (00:00) or a time selected by a user.

According to an embodiment, the processor 520 may configure the second reference time as a time after a predetermined time has elapsed from midnight, and for example, the second reference time may be configured as “00:30”.

According to an embodiment, the processor 520 may configure the second reference time as a sleep start time of a user's sleep time.

According to an embodiment, the processor 520 may store sleep time information, alarm setting information, and/or schedule information as user activity record information.

According to an embodiment, the processor 520 may collect a sleep pattern through an application (for example, a health application) related to a user's sleep installed in the electronic device 501 or an external electronic device (for example, a wearable electronic device) connected to communication with the electronic device and store sleep time information.

According to an embodiment, the processor 520 may collect schedule information and/or alarm setting patterns and/or periods through an application (for example, a calendar application and/or an alarm application) related to user activities installed in the electronic device 501 and store alarm setting information and/or schedule information.

For example, the processor 520 may identify that the first condition for changing the date is satisfied in the situation shown in [Table 1] below.

TABLE 1

| | |
|---|---|
| Time when user utterance is | 00:10 on Jun. 2, 2022 |
| obtained | |
| First reference time-second | 00:00 to 00:30 |
| reference time | |
| User utterance | Set the alarm for tomorrow at 9 o'clock |

As shown in [Table 1], when a time range of the first reference time and the second reference time is “midnight (00:00) to 00:30” and a user utterance of “set the alarm for tomorrow at 9 o'clock” is acquired at “00:10 on Jun. 2, 20222”, the date and time when the user utterance is obtained (June 02, 00:10:00) pass midnight (00:00), which is the first reference time and are included in the time range (midnight (00:00 to 00:30) of the first reference time and the second reference time, and thus the processor 520 may identify that the date and time detected in the user utterance (June 02, 00:10:00) satisfy the first condition for changing the date. According to an embodiment, when the date and time detected in the user utterance are interpreted as a plurality of dates and times, the processor 520 may identify that the second condition for changing the time change is satisfied.

According to an embodiment, the processor 520 may identify the user's sleep time, based on the user's activity record information, and when the time detected in the user utterance is included in the user's sleep time, whether the time detected in the user utterance is included in the user's activity time.

According to an embodiment, the processor 520 may identify the user's activity time, based on the user's activity record information, and when the time detected in the user utterance is not included in the user's activity time in the state where the time detected in the user utterance is included in the user's sleep time, may identify that the user's activity record information satisfies the second condition for changing the time.

According to an embodiment, the processor 520 may identify the user's schedule information and/or a time set in the user's alarm information as the user's activity time, based on the user's activity record information.

For example, the processor 520 may identify that the second condition for changing the time is satisfied in the situation shown in [Table 2] below.

TABLE 2

| | |
|---|---|
| Time when user utterance is | 00:10 on Jun. 2, 2022 |
| obtained | |
| Sleep time | 00:30 to 8:00 a.m. |
| User utterance | Remind me of shopping at 2 o'clock |

As shown in [Table 2], when acquiring the user utterance of “Please remind me of shopping at 2 o'clock” at 00:10 on Jun. 2, 2022, the processor 520 may identify that 2:00 a.m. that is the closest to the current time is included in the user's sleep time of “00:30 a.m. to 8:00 a.m.” When the time (for example, 2:00 a.m.) detected in the user utterance is included in the sleep time, the processor 520 may identify that the time (for example, 2:00 a.m.) detected in the user utterance satisfies the second condition for changing the time if the user's activity time (for example, schedule information and/or alarm information) including the time (for example, 2:00 a.m.) detected in the user utterance does not exist. For example, the processor 520 may identify that the second condition for changing the time is not satisfied in the situation shown in [Table 3] below.

TABLE 3

| | |
|---|---|
| Time when user utterance is | 00:10 on Jun. 2, 2022 |
| obtained | |
| Sleep time | 00:30 to 8:00 a.m. |
| Activity time | Alarm is set for 2 a.m. |
| User utterance | Remind me of shopping at 2 o'clock |

As shown in [Table 3], when acquiring the user utterance of “Please remind me of shopping at 2 o'clock” at 00:10 on Jun. 2, 2022, the processor 520 may identify that 2:00 a.m. that is the closest to the current time is included in the user's sleep time of “00:30 a.m. to 8:00 a.m.” When the time (2 a.m.) detected in the user utterance is included in the sleep time, the processor 520 may identify that the time detected in the user utterance (2:00 a.m.) does not satisfy the second condition for changing the time. For example, the processor 520 may confirm that if user's activity time (“alarm is set for 2:00 a.m.”) including the time (2:00 a.m.) detected in the user utterance exists. For example, the processor 520 may identify that the second condition for changing the time is not satisfied in the situation shown in [Table 4] below.

TABLE 4

| | |
|---|---|
| Time when user | 00:10 on Jun. 2, 2022 |
| utterance is | |
| obtained | |
| Sleep time | 11 p.m. to 8 a.m. |
| Activity time | Early morning exercise schedule is set for 6 a.m. |
| User utterance | Set alarm at 6 o'clock |

As shown in [Table 4], when acquiring the user utterance of “Set alarm at 6 o'clock” at “00:10 on Jun. 2, 2022” the processor 520 may identify that 6:00 a.m. that is the closest to the current time is included in the user's sleep time of “11:00 p.m. to 8:00 a.m.” When the time detected in the user utterance (2:00 a.m.) is included in the sleep time, the processor 520 may identify that the time (2:00 a.m.) detected in the user utterance does not satisfy the second condition for changing the time if the user's activity time (“Early morning exercise schedule is set for 6:00 a.m.”) including the time detected in the user utterance (2:00 a.m.) exists. For example, the processor 520 may identify that the second condition for changing the time is not satisfied in the situation shown in [Table 5] below.

TABLE 5

| | |
|---|---|
| Time when user utterance is | 00:10 on Jun. 2, 2022 |
| obtained | |
| Sleep time | 10 p.m. to 7 a.m. |
| Activity time | Alarm is set for 7 a.m. every day |
| User utterance | Set alarm at 7 a.m. |

As shown in [Table 5], when acquiring the user utterance of “Set alarm at 7:00 a.m.” at “00:10 on Jun. 2, 2022” the processor 520 may identify that 7:00 a.m. that is the closest to the current time is included in the user's sleep time of “10:00 p.m. to 7:00 a.m.” When the time (7:00 a.m.) detected in the user utterance is included in the sleep time, the processor 520 may identify that the time detected in the user utterance (7:00 a.m.) does not satisfy the second condition for changing the time if the user's activity time (“Alarm is set for 7:00 a.m. every day) including the time (for example, 7:00 a.m.) detected in the user utterance exists. According to an embodiment, the processor 520 may change the date detected in the user utterance if the date and time when the user utterance is obtained satisfy the first condition for changing the date, and may change the time detected in the user utterance if the user's activity record information satisfies the second condition for changing the time.

According to an embodiment, if the date and time when the user utterance is obtained satisfy the first condition for changing the date, the processor 520 may change the date detected in the user utterance to a date one day before (for example, date −=1).

According to an embodiment, when the user's activity record information satisfies the second condition for changing the time, the processor 520 may change the time to a time (for example, time+=12) obtained by adding 12 hours to the time detected in the user utterance.

For example, the processor 520 may change the date by satisfying the first condition for changing the date in the situation shown in [able 6] below.

TABLE 6

| | |
|---|---|
| Time when user utterance is | 00:10 on Jun. 2, 2022 |
| obtained | |
| First reference time-second | 00:00 to 00:30 |
| reference time | |
| Sleep time | 00:30 to 8:00 a.m. |
| User utterance | Set alarm at 9:00 tomorrow |

As shown in [Table 6], since the user utterance time has elapsed midnight (00:00) that is the first reference time and is included in the time range midnight (00:00) to 00:30 of the first reference time and the second reference time and thus the date and time (Jun. 2, 2022, 00:10) when the user utterance is obtained satisfy the first condition for changing the date, the processor 520 may change the date to “Jun. 2, 2022 that is a day before “Jun. 3, 2022” corresponding to “tomorrow”, that is the date detected in the user utterance. As 9:00 a.m. that is the time detected in the user utterance is not included in the sleep time and thus the second condition is not satisfied and 9:00 a.m. is maintained, the processor 520 may change the date for performing the function of the user utterance from “9:00 a.m. on Jun. 3, 2022” to “9:00 a.m. on Jun. 2, 2022”. For example, the processor 520 may change the date and time by satisfying the first condition for changing the date and the second condition for changing the time in the situation shown in [Table 7] below.

TABLE 7

| | |
|---|---|
| Time when user utterance is | 00:10 on Jun. 2, 2022 |
| obtained | |
| First reference time- | 00:00 to 00:30 |
| second reference time | |
| Sleep time | 00:30 to 8:00 a.m. |
| User utterance | Remind me of shopping at 2:00 |

As shown in [Table 7], since the user utterance time has elapsed midnight (00:00) that is the first reference time and is included in the time range midnight (00:00) to 00:30 of the first reference time and the second reference time and thus the date and time (Jun. 2, 2022, 00:10) when the user utterance is obtained satisfy the first condition for changing the date, the processor 520 may change “Jun. 3, 2022” corresponding to “tomorrow” that is the date detected in the user utterance to “Jun. 2, 2022 that is a day before. Since 2:00 a.m. that is the time detected in the user utterance is included in the sleep time and the user's activity time (for example, schedule information and/or alarm information) including the time detected in the user utterance (for example, 2:00 a.m.) does not exist and thus the second condition for changing the time (for example, 2:00 a.m.) detected in the user utterance is satisfied, the processor 520 may change the date to “2:00 p.m. by adding 12 hours to 2:00. a.m. detected in the user utterance. The processor 520 may change the date for performing the function of the user utterance from “2:00 p.m. on Jun. 3, 2022” to “2:00 p.m. on Jun. 2, 2022”. For example, the processor 520 may change the date for performing the function of the user utterance from “2024” to “2023” when the first reference time—the second reference time is “00:00 to 00:30 on Jan. 1, 2023” and the user utterance of “Tell me next year's holiday” is detected at “00:10 on Jan. 23, 2023”.

For example, the processor 520 can change the date for performing the function of the user utterance from “next week” to “this week” if the first reference time—the second reference time is “00:00 to 00:30 on Monday” and the user utterance of “Tell me may schedule for next week” is detected at “00:10 on Monday.”

According to an embodiment, the processor 520 may output, through the output module 570, a message recommending performance of a function on the date and/or time changed from the date and time detected in the user utterance.

According to an embodiment, when the date and time when the user utterance is obtained satisfy at least one of the first condition for changing the date and the user's activity record information satisfies at least one of the second conditions for changing the time, the processor 520 may change the date and/or time detected in the user utterance, output a message recommending performance of a function on the changed date and time through the output module 570, and when a user input for agreeing to perform the function on the changed date and time is identified, process the performance of the function on the changed date and time.

According to an embodiment, the processor 520 may output, through the output module 570, a message recommending performance of a function on the date and/time changed from the date and time detected in the user utterance and a message recommending performance of a function on the date and time detected in the user utterance. The processor 520 may process performance of the function on the changed date and time when the user input for agreeing to perform the function on the changed date and time is identified, and process performance of the function on the date and time detected in the user utterance when the user input for agreeing to perform the function on the date and time detected in the user utterance is identified.

According to an embodiment, the processor 520 may output a message recommending performance of a function by voice through the speaker of the output module 570, or display the message recommending the performance of the function through the display of the output module 570.

For example, <Table 8> below describes the operation of interpreting the date and time detected in the user utterance when the date and time when the user utterance is obtained are Jun. 2, 2022, 00:30” and the sleep time is “01:00 p.m. to 08:00 a.m.”

TABLE 8

| | Analyze date | | |
|---|---|---|---|
| | and time | Analyze | |
| User | detected in | changed date | |
| utterance | user utterance | and time | Analysis result |
| Tomorrow | 3rd | 2nd | weather on the 3rd- weather |
| weather | | | on the 2nd |
| Today | 2nd | X | weather on the 2nd |
| weather | | | |
| Alarm at | 9:00 a.m. on the | 9:00 a.m. | 9:00 a.m. on the 3rd - 9:00 |
| 9:00 | 3^{rd} | on the 2nd | a.m. on the 2nd |
| tomorrow | | | |
| Alarm at | 3:00 a.m. on the | 3:00 p.m. | 3:00 a.m. on the 2nd - 3:00 |
| 3:00 | 3^{rd} | on the 2nd | a.m. on the 3rd |
| tomorrow | | | |
| Alarm at | 3:00 a.m. on the | 3:00 p.m. | 3:00 p.m. on the 2nd - 3:00 |
| 3:00 today | 2^{nd} | on the 2nd | a.m. on the 2nd |
| Alarm at | 3:00 a.m. on the | X | 3:00 a.m. on the 3rd |
| 3:00 a.m. | 3rd | | |
| on the 3rd | | | |
| Alarm at | 3:00 p.m. on the | X | 3:00 p.m. on the 2nd |
| 15:00 on | 2nd | | |
| the 2nd | | | |

According to an embodiment, the input module 510 may be implemented substantially the same as or similar to the input module 150 of FIG. 1. According to an embodiment, the input module 510 may include a microphone.

According to an embodiment, an utterance processing module 530 may analyze and/or interpret phrases in the user utterance obtained through the input module 150.

According to an embodiment, the utterance processing module 530 may identify whether the user utterance is a user utterance related to a date and time, and identify whether the user utterance is a user utterance for performing a function.

According to an embodiment, the output module 570 may include a display and a speaker.

According to an embodiment, the display of the output module 570 may be implemented substantially the same as or similar to the display module 160 of FIG. 1.

According to an embodiment, the speaker of the output module 570 may be implemented substantially the same as or similar to the sound output module 155 of FIG. 1.

According to an embodiment, the output module 570 may output a message recommending performance of a function on a date and time changed from the date and time detected in the user utterance and/or a message recommending performance of a function on the date and time detected in the user utterance.

According to an embodiment, the communication module 590 may be implemented substantially the same as or similar to the communication module 190 of FIG. 1, and may include a plurality of communication circuits using different communication technologies.

According to an embodiment, the first communication module 390 may include at least one of a wireless LAN module (not shown) and a short-range communication module (not shown), and the short-range communication module (not shown) may include a ultra-wideband (UWB) communication module, a Wi-Fi communication module, an NFC communication module, a Bluetooth legacy communication module, and/or a BLE communication module.

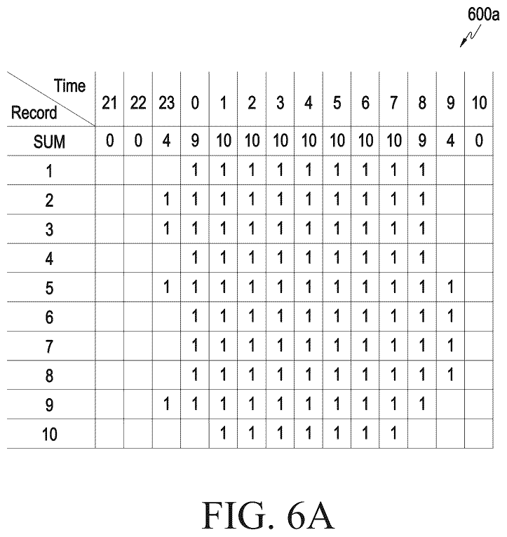

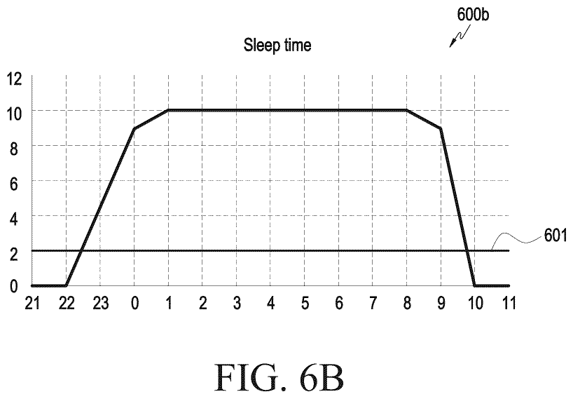

FIGS. 6A and 6B are diagrams 600a to 600b illustrating an operation of setting a sleep time in an electronic device according to an embodiment.

Referring to FIG. 6A, when making a connection to communicate with the electronic device and receiving the user's sleep time from an external electronic device (for example, a wearable electronic device) worn by the user, the electronic device (for example, the electronic device 101 of FIG. 1, the user terminal 290 of FIG. 2, and/or the electronic device 501 of FIG. 5) may record the user's sleep time during a predetermined period and calculate the sum.

Referring to FIG. 6B, the electronic device (for example, the electronic device 101 of FIG. 1, the user terminal 290 of FIG. 2, and/or the electronic device 501 of FIG. 5) may set a start time of sleep (for example, 22:00) and an end time of sleep (for example, 9:30 a.m.), based on a threshold value 601 by using a sleep record for a predetermined time as shown in FIG. 6A.

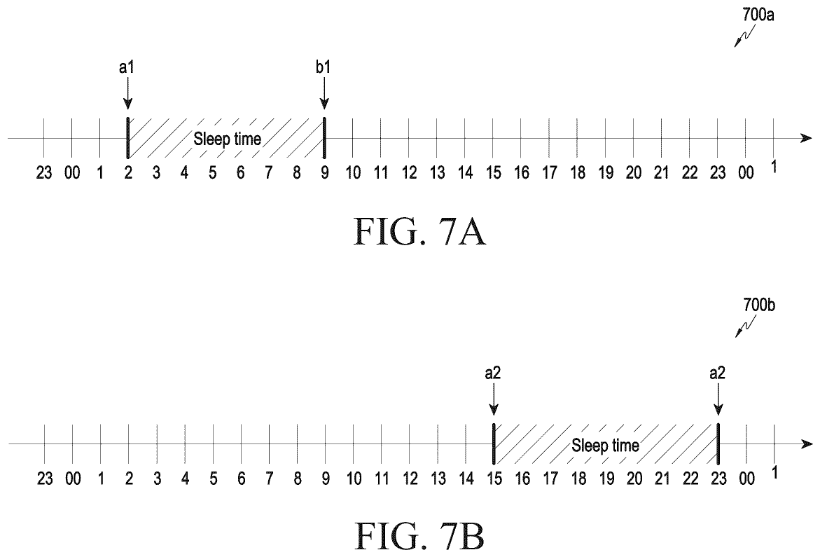

FIGS. 7A to 7B are diagrams 700a to 700b illustrating an operation of activating a function of changing a date and time in an electronic device according to an embodiment.

When receiving a user utterance related to the date and time through an input module of the electronic device (for example, the input module 510 of FIG. 5), the electronic device (for example, the electronic device 101 of FIG. 1, the user terminal 290 of FIG. 2, and/or the electronic device 501 of FIG. 5) may determine whether to activate the function of changing the date and time by applying the user's sleep time to the <Equation 1>.

Referring to FIG. 7A, when the sleep start time a1 is 2:00 a.m. and the sleep end time b1 is 9:00 a.m., the function of changing the date and time may be activated by satisfying a condition such that “2<9” from min(2, 24th-2=22)<min(9, 24:00−9=15) to 2<9 by applying the <Equation 1>.

Referring to FIG. 7B, when the sleep start time a2 is 15:00 and the sleep end time b2 is 23:00., the function of changing the date and time may be activated by satisfying a condition such that “9>1” from min(15, 24th-15=9)<min(123, 24th-23=1) by applying the <Equation 1>.

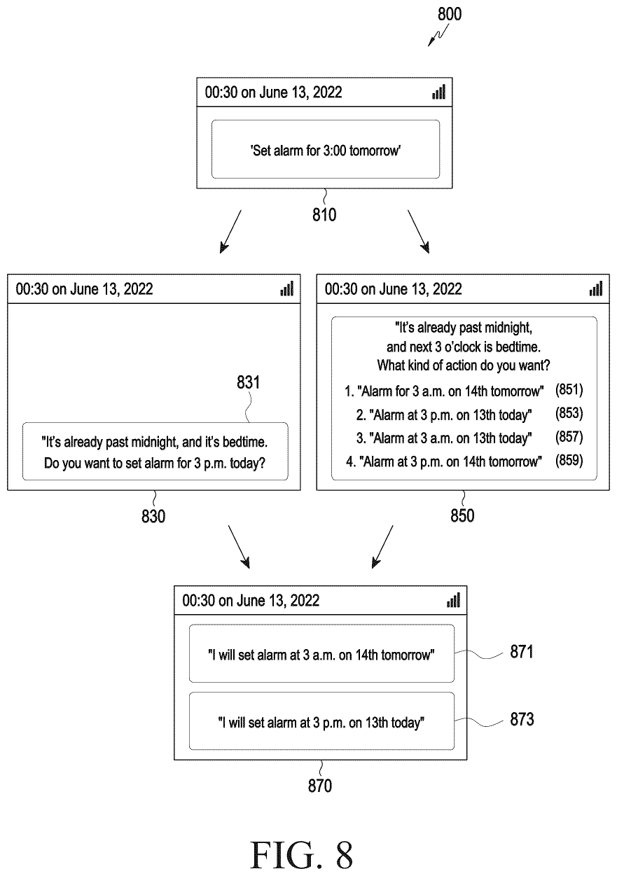

FIG. 8 is a diagram 800 illustrating an operation of processing a user utterance in an electronic device according to an embodiment.

Referring to FIG. 8, as shown on a screen 810, when acquiring a user utterance of “Set alarm at 9 o'clock tomorrow” at 00:30 on Jun. 13, 2022”, the electronic device (for example, the electronic device 101 in FIG. 1, the user terminal 290 in FIG. 2, and/or the electronic device 501 in FIG. 5) may change “Jun. 14, 2022” corresponding to “tomorrow” that is the date detected in the user utterance to “Jun. 13, 2022,” that is a day before since the time at which the user utterance is obtained passes midnight (00:00) that is a first reference time and is included in a time range (for example, midnight (00:00) to 00:30) of the first reference time and a second reference time, and thus the date and time (Jun. 13, 2022, 00:30) detected in the user utterance satisfy a first condition for changing the date.

Since 3:00 a.m. that is the time detected in the user utterance is included in the sleep time (for example, 00:30 a.m. to 8:00 a.m.) and user's activity time (for example, schedule information and/or alarm information) detected in the user utterance including 3:00 a.m. that is the time detected in the user utterance does not exist, the electronic device may change the time (for example, 3:00 a.m.) detected in the user utterance to “3:00 p.m.” by adding 12 hours to 3:00 a.m.” by satisfying the second condition for changing the time. The electronic device may change the date for performing the function of the user utterance from “3:00 a.m. on Jun. 14, 2022” to “3:00 p.m. on Jun. 13, 2022”.

As shown on a screen 830, the electronic device (for example, the electronic device 101 of FIG. 1, the user terminal 290 of FIG. 2, and/or the electronic device 501 of FIG. 5) may output a recommendation message 831 for setting an alarm at 3:00 p.m. today (Jun. 13, 2022) through a speaker or display of an output module (for example, the output module 570 of FIG. 5) while notifying that the midnight has elapsed.

As shown on a screen 850, the electronic device (for example, the electronic device 101 of FIG. 1, the user terminal 290 of FIG. 2, and/or the electronic device 501 of FIG. 5) may allow the user to select performance of a desired function among a plurality of recommendation messages for setting the alarm while notifying that the midnight has elapsed through the speaker or display of the output module (for example, the output module 570 of FIG. 5).

The plurality of recommendation messages for setting the alarm may include a recommendation message 851 for setting an “alarm at 3:00 a.m. on the 14th tomorrow” without changing the date and time detected in the user utterance, a recommendation message 853 for setting an “alarm at 3:00 p.m. on the 13th today” after changing the date and time detected in the user utterance, a recommendation message 855 for setting an “alarm at 3:00 a.m. on the 13th today” without changing the date detected in the user utterance, and a recommendation message 857 for setting an “alarm at 3:00 p.m. on the 14th tomorrow” without changing the date detected in the user utterance.

As shown on a screen 870, when the user does not agree with the recommendation message 831 output on the screen 830, or when the user selects the recommendation message 853 from among a plurality of recommendation messages on the screen 850, the electronic device (for example, the electronic device 101 of FIG. 1, the user terminal 290 of FIG. 2, and/or the electronic device 501 of FIG. 5) may output a result message 871 of notifying that a notification has been set for 3:00 a.m. on the 14^{th} tomorrow through the speaker or display of the output module (for example, the output module 570 of FIG. 5). When the user agrees with the recommendation message 831 output on the screen 830 or the user selects the recommendation message 853 from among the plurality of recommendation messages on the screen 850, the electronic device may output a result message 873 of notifying that a notification has been set for 3 p.m. on the 13rd today through the speaker or display of the output module (for example, the output module 570 of FIG. 5).

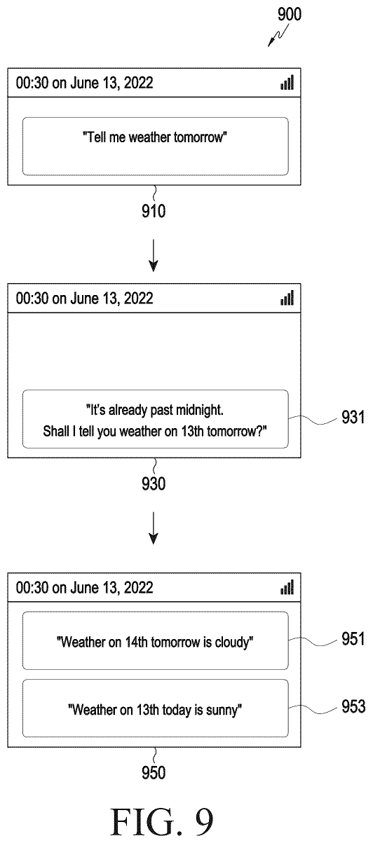

FIG. 9 is a diagram 900 illustrating an operation of processing a user utterance in an electronic device according to an embodiment.

Referring to FIG. 9, as shown on a screen 910, when acquiring a user utterance of “Tell me the date tomorrow” at “00:30 on Jun. 13, 2022”, the electronic device (for example, the electronic device 101 in FIG. 1, the user terminal 290 in FIG. 2, and/or the electronic device 501 in FIG. 5) may change “Jun. 14, 2022” corresponding to “tomorrow” that is the date detected in the user utterance to “Jun. 13, 2022,” that is a day before since the time at which the user utterance is obtained passes midnight (00:00) that is a first reference time and is included in a time range (for example, midnight (00:00) to 00:30) of a first reference time and a second reference time, and thus the date and time (Jun. 13, 2022, 00:30) detected in the user utterance satisfy a first condition for changing the date.

As shown on a screen 930, the electronic device (for example, the electronic device 101 of FIG. 1, the user terminal 290 of FIG. 2, and/or the electronic device 501 of FIG. 5) may output a recommendation message 931 indicating provision of information on the weather today (Jun. 13, 2022) through the speaker or display of the output module (for example, the output module 570 of FIG. 5) while notifying that the midnight has elapsed.

As shown on a screen 950, when the user does not agree with a recommendation message 931 output on the screen 930, the electronic device (e.g., the electronic device 101 of FIG. 1, the user terminal 290 of FIG. 2, and/or the electronic device 501 of FIG. 5) may output a result message 951 of providing weather information for the 14^{th} tomorrow through the speaker or display of the output module (for example, the output module 570 of FIG. 5). When the user agrees with the recommendation message 831 output on the screen 930, the electronic device may output a result message 953 of providing weather information for the 13rd today through the speaker or display of the output module (for example, the output module 570 of FIG. 5).

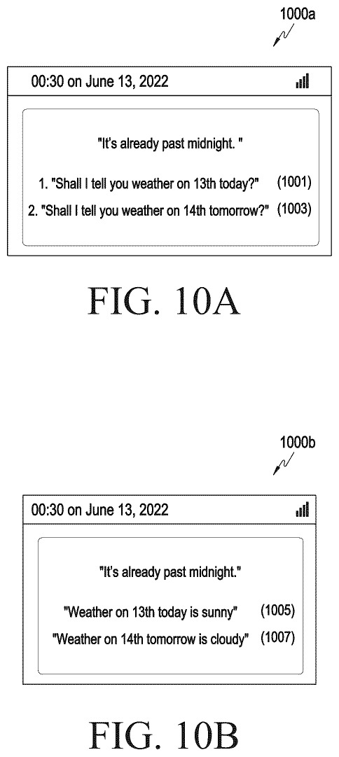

FIGS. 10A to 10B are diagrams 1000a to 1000b illustrating an operation of processing a user utterance in an electronic device according to an embodiment.

When acquiring a user utterance of “Tell me the date tomorrow” at “00:30 on Jun. 13, 2022”, the electronic device (for example, the electronic device 101 in FIG. 1, the user terminal 290 in FIG. 2, and/or the electronic device 501 in FIG. 5) may change “Jun. 14, 2022” corresponding to “tomorrow” that is the date detected in the user utterance to “Jun. 13, 2022,” that is a day before since the time at which the user utterance is obtained passes midnight (00:00) that is a first reference time and is included in a time range (for example, midnight (00:00) to 00:30) of a first reference time and a second reference time, and thus the date and time (Jun. 13, 2022, 00:30) detected in the user utterance satisfy a first condition for changing the date.

As shown in FIG. 10A, the electronic device (for example, the electronic device 101 of FIG. 1, the user terminal 290 of FIG. 2, and/or the electronic device 501 of FIG. 5) may output a recommendation message 1001 indicating provision of information on the weather today (Jun. 13, 2022) and a recommendation message 1003 indicating provision of information on the weather tomorrow (Jun. 14, 2022) through the speaker or display of the output module (for example, the output module 570 of FIG. 5) so as to allow the user to perform selection.

As shown in FIG. 10B, the electronic device (for example, the electronic device 101 of FIG. 1, the user terminal 290 of FIG. 2, and/or the electronic device 501 of FIG. 5) may output a result message 1005 that provides weather information on the 13th today and a result message 1007 that provides weather information on the 14th tomorrow while notifying that midnight has passed through the speaker or display of the output module (for example, the output module 570 of FIG. 5).

According to an embodiment, the electronic device 101 (FIG. 1, 290 of FIG. 2, or 501 of FIG. 5) may include an input module 150 of FIG. 1, 295 of FIG. 2, or 510 of FIG. 5, an output module 155 of FIG. 1, 160 of FIG. 1, 294 of FIG. 2, or 570 of FIG. 5, and a processor 120 of FIG. 1, 292 of FIG. 2, or 520 of FIG. 5.

According to an embodiment, the processor (120 of FIG. 1, 292 of FIG. 2, or 520 of FIG. 5), when a user utterance related to a date and time acquired through the input module is identified as a user utterance for performing a function, may identify whether the date and time detected in the user utterance are interpreted as a plurality of dates and times, based on a date and time when the user utterance is obtained.

According to an embodiment, the processor (120 of FIG. 1, 292 of FIG. 2, or 520 of FIG. 5), when the date and time detected in the user utterance are interpreted as the plurality of dates and times, may change the date detected in the user utterance if the date and time when the user utterance is obtained satisfy a first condition for changing the date, and change the time detected in the user utterance if user's activity record information satisfies a second condition for changing the time.

According to an embodiment, the processor (120 of FIG. 1, 292 of FIG. 2, or 520 of FIG. 5) may recommend performance of the function on the changed date or time through the output module.

According to an embodiment, the processor (120 of FIG. 1, 292 of FIG. 2, or 520 of FIG. 5) may identify a user's sleep time, based on the user's activity record information and, when a difference between a sleep end time and a first reference time is smaller than a difference between a sleep start time and the first reference time, activate a function of changing the date and time.

According to an embodiment, the processor (120 of FIG. 1, 292 of FIG. 2, or 520 of FIG. 5), when the function of changing the date and time is activated, may identify whether the date and time detected in the user utterance are interpreted as a plurality of dates and times.

According to an embodiment, the processor (120 of FIG. 1, 292 of FIG. 2, or 520 of FIG. 5), when the date and time when the user utterance is acquired are included in a range of the first reference time and a second reference time in a state where the date and time when the user utterance is obtained are identified as a time having elapsed the first reference time, may identify that the first condition for changing the date is satisfied.

According to an embodiment, the processor (120 of FIG. 1, 292 of FIG. 2, or 520 of FIG. 5) may set the first reference time as midnight and set the second reference time as a time after a predetermined time from the midnight or the sleep start time.

According to an embodiment, the processor (120 of FIG. 1, 292 of FIG. 2, or 520 of FIG. 5), when it is identified that the time detected in the user utterance is included in the user's sleep time and is not included in the user's activity time, based on the user's activity record information, may identify that the second condition for changing the time is satisfied.

According to an embodiment, the processor (120 of FIG. 1, 292 of FIG. 2, or 520 of FIG. 5) may store at least one piece of sleep time information, alarm setting information, or scheduling information as the user's activity record information.

According to an embodiment, the processor (120 of FIG. 1, 292 of FIG. 2, or 520 of FIG. 5), when the date and time when the user utterance is obtained satisfy the first condition for changing the date, may change the date detected in the user utterance to a date one day before.

According to an embodiment, the processor (120 of FIG. 1, 292 of FIG. 2, or 520 of FIG. 5), when the user's activity record information satisfies the second condition for changing the time, may change the time to a time obtained by adding 12 hours to the time detected in the user utterance.

According to an embodiment, the processor (120 of FIG. 1, 292 of FIG. 2, or 520 of FIG. 5) may output a message recommending performance of the function on the changed date and time through the output module.

According to an embodiment, the processor (120 of FIG. 1, 292 of FIG. 2, or 520 of FIG. 5) may output the message recommending performance of the function on the changed date and time and a message recommending performance of the function on the date and time detected in the user utterance through the output module.

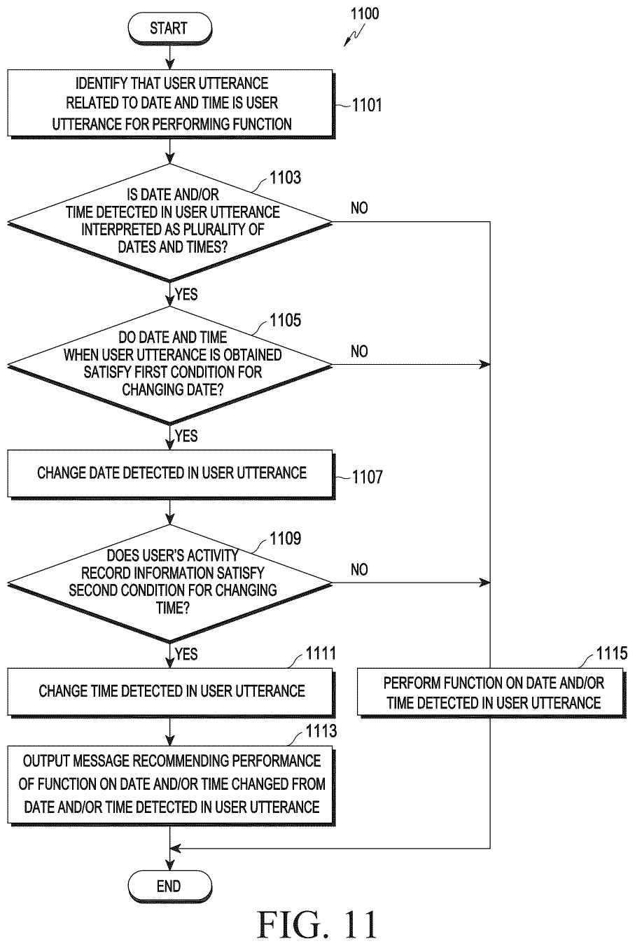

FIG. 11 is a flowchart 1100 illustrating an operation of processing a user utterance in a wearable electronic device according to an embodiment. Operations of processing the user utterance may include operations 1101 to 1115. In the following embodiment, respective operations may be sequentially performed, but the sequential performance is not necessary. For example, the order of each operation may be changed, at least two operations may be performed in parallel, or other operations may be added.

In operation 1101, the electronic device (for example, the electronic device 101 of FIG. 1, the user terminal 290 of FIG. 2, and/or the electronic device 501 of FIG. 5) may identify a user utterance related to a date and time as a user utterance for performing a function.

According to an embodiment, an input module (for example, the input module 510 of FIG. 5) of the electronic device may acquire the user utterance.

According to an embodiment, based on a result of syntactic analysis of the user utterance through an utterance processing module (for example, the utterance processing module 530 of FIG. 5) of the electronic device, it may be identified that the user utterance is a user utterance related to a date and time, and the user utterance related to the date and time is a user utterance for performing a function.

In operation 1103, the electronic device (for example, the electronic device 101 of FIG. 1, the user terminal 290 of FIG. 2, and/or the electronic device 501 of FIG. 5) may determine whether the date and/or time detected in the user utterance is interpreted as a plurality of dates and times.

According to an embodiment, when receiving a user utterance through an input module (for example, the input module 510 of FIG. 5), the electronic device may identify the user's sleep time, based on user's activity record information, and when the difference between an end time of sleep and a first reference time is smaller than the difference between the start time of sleep and a first reference, may activate a function for changing the date and time. When the function for changing the date and time is activated, the electronic device may identify whether the date and time detected in the user utterance are interpreted as a plurality of dates and times, based on the time when the user utterance is obtained.

According to an embodiment, the electronic device may activate the function for changing the date and time when the condition of <Equation 1> is satisfied, and may deactivate the function for changing the date and time when the condition of <Equation 1> is not satisfied.

According to an embodiment, the electronic device may identify whether the date and/or time detected in the user utterance is interpreted as a plurality of dates and times, based on the date and time when the user utterance is obtained.

According to an embodiment, when the date and time when the user utterance is obtained have passed midnight, the date detected in the user utterance is not designated as a number (for example., tomorrow), and the time detected in the user utterance is not designated as the morning or afternoon, the electronic device may determine that the date and/or time is interpreted as a plurality of dates and times.

When it is determined that the date and/or time detected in the user utterance is interpreted as a plurality of dates and times in operation 1103, the electronic device may identify whether the date and time on which the user's utterance is obtained satisfy the first condition for changing the date in operation 1105.

According to an embodiment, when the date and time when the user utterance is obtained satisfy “the first reference time <=the date and time when the user utterance is obtained <=the second reference time” in the state where the date and time when the user utterance is obtained have passed the first condition, the electronic device may identify that the date and time when the user utterance is obtained satisfy the first condition for changing the date and time when the user utterance is obtained.

According to an embodiment, the electronic device may set the first reference time as a default time (for example, midnight (00:00) or a time selected by the user.

According to an embodiment, the electronic device may set the second reference time as a time after a predetermined time from midnight and, for example, as 00:30”.

According to an embodiment, the electronic device set the second reference time as a sleep start time of the user's sleep time.

According to an embodiment, the electronic device may store sleep time information, alarm setting information, and/or schedule information as user's activity record information.

When the date and time when the user utterance is obtained satisfy the first condition for changing the date in operation 1105, the electronic device may change the date detected in the user utterance in operation 1107.

According to this configuration, when the date and time when the user utterance is obtained satisfy the first condition for changing the date, the electronic device may change the date detected in the user utterance to a date one day before (for example, date−=1).

In operation 1109, the electronic device (for example, the electronic device 101 of FIG. 1, the user terminal 290 of FIG. 2, and/or the electronic device 501 of FIG. 5) may identify whether the user's activity record information satisfies the second condition for changing the time.

According to an embodiment, the electronic device may identify the user's sleep time, based on the user's activity record information, and when the time detected in the user utterance is included in the user's sleep time, may identify whether the time detected in the user utterance is included in the user's activity time. When the time detected in the user utterance is not included in the user's activity time in the state where the time detected in the user utterance is included in the user's sleep time, the electronic device may identify that the user's activity record information satisfies the second condition for changing the time.

According to an embodiment, the electronic device may identify a time set in the user's schedule information and/or the alarm information as the user's activity time, based on the user's activity record information.

When the user's activity record information satisfies the second condition for changing the time in operation 1109, the electronic device may change the time detected in the user utterance in operation 1111.

According to an embodiment, when the user's activity record information satisfies the second condition for changing the time, the electronic device may change the time to a time (for example, time+=12) obtained by adding 12 hours to the time detected in the user utterance.

In operation 1113, the electronic device (for example, the electronic device 101 of FIG. 1, the user terminal 290 of FIG. 2, and/or the electronic device 501 of FIG. 5) may output a message recommending performance of a function on a date and/or time changed from the date and/or time detected in the user utterance.

According to an embodiment, the message recommending performance of the function on the changed date and/or time may be output through an output module (for example, the output module 570 of FIG. 5).

According to an embodiment, the processor 520 may output the message recommending performance of the function on the date and time detected in the user utterance together with the message recommending performance of the function on the date and/or time changed from the date and/or time detected in the user utterance through the output module 570.

According to an embodiment, the electronic device may output the message recommending performance of the function by voice through a speaker of the output module or display the message recommending performance of the function through a display of the output module.

When the date and/or time detected in the user utterance is not interpreted as a plurality of dates and/or times in operation 1103, the date and time when the user utterance is obtained does not satisfy the first condition for changing the date in operation 1105, or the user activity record information does not satisfy the second condition for changing the time in operation 1109, or the electronic device may perform the function on the date and/or time detected in the user utterance in operation 1115.

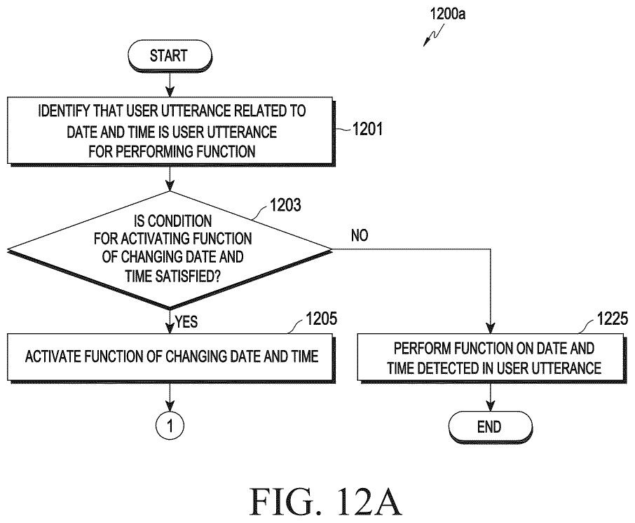

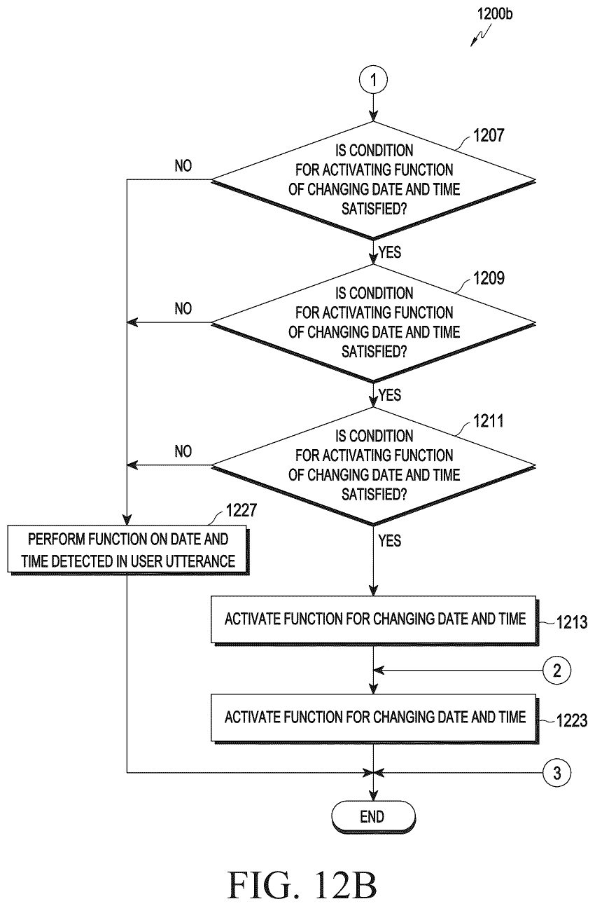

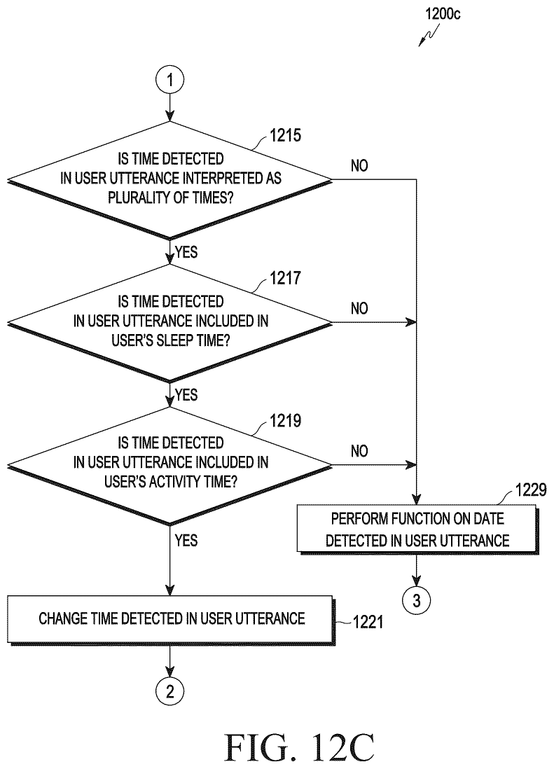

FIGS. 12A to 12C are flowcharts 1200a to 1200c illustrating an operation of processing a user utterance in a wearable electronic device according to an embodiment. Operations of processing the user utterance may include operations 1201 to 1229. In the following embodiment, respective operations may be sequentially performed, but the sequential performance is not necessary. For example, the order of each operation may be changed, at least two operations may be performed in parallel, or other operations may be added.

In operation 1201, the electronic device (for example, the electronic device 101 of FIG. 1, the user terminal 290 of FIG. 2, and/or the electronic device 501 of FIG. 5) may identify a user utterance related to a date and time as a user utterance for performing a function.

According to an embodiment, an input module (for example, the input module 510 of FIG. 5) of the electronic device may acquire the user utterance.

According to an embodiment, based on a result of syntactic analysis of the user utterance through an utterance processing module (for example, the utterance processing module 530 of FIG. 5) of the electronic device, it may be identified that the user utterance is a user utterance related to a date and time, and the user utterance related to the date and time is a user utterance for performing a function.

In operation 1203, the electronic device (for example, the electronic device 101 of FIG. 1, the user terminal 290 of FIG. 2, and/or the electronic device 501 of FIG. 5) may determine whether a condition for activating the function of changing the date and time is satisfied.

According to an embodiment, the electronic device may identify the user's sleep time, based on the user's activity record information, and when the difference between an end time of sleep and a first reference time is smaller than the difference between the start time of sleep and a first reference, may identify that the condition for activating the function of changing the date and time is satisfied.

According to an embodiment, when the condition of <Equation 1> is satisfied, the electronic device may identify that the condition for activating the function of changing the date and time is satisfied.

According to an embodiment, the processor 520 may activate the function of changing the date and time when the sleep end time is closer to midnight than the sleep start time through <Equation 1>.

When it is identified that the condition for activating the function of changing the date and time is not satisfied in operation 1203, the electronic device may perform the function on the date and time detected in the user utterance in operation 1225.

When it is identified that the condition for activating the function of changing the date and time is satisfied in operation 1203, the electronic device may activate the function of changing the date and time in operation 1205.

In operation 1207, the electronic device (for example, the electronic device 101 of FIG. 1, the user terminal 290 of FIG. 2, and/or the electronic device 501 of FIG. 5) may identify whether the date detected in the user utterance is interpreted as a plurality of dates.

According to an embodiment, the electronic device may identify whether the date detected in the user utterance is interpreted as a plurality of dates, based on the date and time when the user utterance is obtained.

According to an embodiment, the electronic device may determine that the date detected in the user utterance is interpreted as a plurality of dates when the date detected in the user utterance is not designated as a number (for example, tomorrow), based on the date and/or time detected in the user utterance (for example, 00:10).

When it is determined that the date detected in the user utterance is interpreted as a plurality of dates in operation 1207, the electronic device may identify whether the time when the user utterance is obtained has elapsed the first reference time in operation 1209.

According to an embodiment, the electronic device may set the first reference time as a default time (for example, midnight (00:00) or a time selected by the user.

When it is determined that the time when the user utterance is obtained has elapsed the first reference time in operation 1209, the electronic device may identify whether the date and time when the user utterance is obtained exist within a time range from the first reference time to the second reference time in operation 1211.

According to an embodiment, the electronic device may identify whether the date and time when the user utterance is obtained satisfy “the first reference time <=the date and time when the user utterance is obtained <=the second reference time”.

According to an embodiment, the electronic device may set the second reference time as a time after a predetermined time from midnight and, for example, as “00:30”.

When the date and time when the user utterance is obtained exist within the time range from the first reference time to the second reference time in operation 1211, the electronic device may change the date detected in the user utterance in operation 1213.

According to an embodiment, the electronic device may change the date detected in the user utterance to a date one day before (for example, date −=1).

In operation 1215, the electronic device (for example, the electronic device 101 of FIG. 1, the user terminal 290 of FIG. 2, and/or the electronic device 501 of FIG. 5) may identify whether the time detected in the user utterance is interpreted as a plurality of times.

According to this configuration, the electronic device may identify whether the time detected in the user utterance is interpreted as a plurality of times, based on the date and time when the user utterance is obtained.

According to an embodiment, the electronic device may determine that the time detected in the user utterance is interpreted as a plurality of times when the time detected in the user utterance is not designated as morning or afternoon, based on the date and/or time (for example, 00:10) detected in the user utterance.

When the electronic device determines that the time detected in the user utterance is interpreted as a plurality of times in operation 1215, the electronic device may determine whether the time detected in the user utterance is included in the user's sleep time in operation 1217.

According to this configuration, the electronic device may detect the user's sleep time information, based on the user's activity record information.

When it is identified that the time detected in the user utterance is included in the user's sleep time in operation 1217, the electronic device may identify whether the time detected in the user utterance is included in the user's active time in operation 1219.

According to an embodiment, the electronic device may identify a time set in the user's schedule information and/or the alarm information as the user's activity time, based on the user's activity record information.

When it is identified that the time detected in the user utterance is not included in the user's active time in operation 1219, the electronic device may change the time detected in the user utterance in operation 1221.

According to an embodiment, the electronic device may change the time (for example, time+=12) by adding 12 hours to the time detected in the user utterance.

In operation 1223, the electronic device (for example, the electronic device 101 of FIG. 1, the user terminal 290 of FIG. 2, and/or the electronic device 501 of FIG. 5) may output a message recommending performance of a function on the changed date and/or time.

According to an embodiment, the electronic device may output a message recommending performance of a function performance on the changed date and/or time through a speaker or display of an output module (for example, the output module 570 of FIG. 5).

According to an embodiment, the electronic device may output, through the speaker or display of the output module, the message recommending performance of the function on the date and time detected in a user utterance together with the message recommending performance of the function on the changed date and/or time.

When it is determined that the date detected in the user utterance is not interpreted as a plurality of dates in operation 1207, it is identified that the time when the user utterance is obtained does not elapse the first reference time in operation 1209, or it is identified that the date and time when the user utterance is obtained do not exist within the time range from the first reference time to the second reference time in operation 1211, the electronic device may perform the function on the date detected in the user utterance in operation 1227.

When it is determined that the time detected in the user utterance is not interpreted as a plurality of times in operation 1215, when the time detected in the user utterance is not included in the user's sleep time in operation 1217, or it is identified that the time detected in the user utterance is included in the user's active time in operation 1219, the electronic device may perform the function on the time detected in the user utterance in operation 1229.

Operation 1207 to operation 1213, operation 1215, and operation 1221 may be simultaneously performed, or may be sequentially performed.

According to an embodiment, a method of processing a user utterance by an electronic device may include an operation of, when a user utterance related to a date and time acquired through an input module is identified as a user utterance for performing a function, identifying whether the date and time detected in the user utterance are interpreted as a plurality of dates and times, based on a date and time when the user utterance is obtained.

According to an embodiment, the method may include an operation of, when the date and time detected in the user utterance are interpreted as the plurality of dates and times, changing the date detected in the user utterance if the date and time when the user utterance is obtained satisfy a first condition for changing the date, and changing the time detected in the user utterance if user's activity record information satisfies a second condition for changing the time.

According to an embodiment, the method may include an operation of recommending performance of the function on the changed date or time through an output module.

According to an embodiment, the method may include an operation of identifying a user's sleep time, based on the user's activity record information and, when a difference between a sleep end time and a first reference time is smaller than a difference between a sleep start time and the first reference time, activating a function of changing the date and time.

According to an embodiment, the method may further include an operation of, when the function of changing the date and time is activated, identifying whether the date and time detected in the user utterance are interpreted as a plurality of dates and times.

According to an embodiment, the method may include an operation of, when the date and time when the user utterance is acquired are included in a range of the first reference time and a second reference time in a state where the date and time when the user utterance is obtained are identified as a time having elapsed the first reference time, identifying that the first condition for changing the date is satisfied.

In the method according to an embodiment, the first reference time may be set as midnight, and the second reference time may be set as a time after a predetermined time from the midnight or the sleep start time.

According to an embodiment, the method may include an operation of, when it is identified that the time detected in the user utterance is included in the user's sleep time and is not included in the user's activity time, based on the user's activity record information, identifying that the second condition for changing the time is satisfied.

According to an embodiment, the method may include an operation of storing at least one piece of sleep time information, alarm setting information, or schedule information as the user's activity record information.

According to an embodiment, the method may include an operation of, when the date and time when the user utterance is obtained satisfy a first condition for changing the date, changing the date detected in the user utterance to a date one day before.

According to an embodiment, the method may include an operation of, when the user's activity record information satisfies a second condition for changing the time, changing the time to a time obtained by adding 12 hours to the time detected in the user utterance.

According to an embodiment, the method may include an operation of outputting a message recommending performance of a function on the changed date and time through the output module.

According to an embodiment, the method may further include an operation of outputting the message recommending performance of the function on the changed date and time and a message recommending performance of the function on the date and time detected in the user utterance through the output module.

According to an embodiment of the disclosure, a non-volatile storage medium storing instructions is provided. The instructions may be configured to cause, when executed by an electronic device, the electronic device to perform at least one operation. The at least one operation may include an operation of, when a user utterance related to a date and time acquired through an input module is identified as a user utterance for performing a function, identifying whether the date and time detected in the user utterance are interpreted as a plurality of dates and times, based on a date and time when the user utterance is obtained, an operation of, when the date and time detected in the user utterance are interpreted as the plurality of dates and times, changing the date detected in the user utterance if the date and time when the user utterance is obtained satisfy a first condition for changing the date, and changing the time detected in the user utterance if user's activity record information satisfies a second condition for changing the time, and an operation of recommending performance of the function on the changed date or time through an output module.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

An embodiment as set forth herein may be implemented as software (e.g., the program 140) including one or more instructions that are stored in a storage medium (e.g., internal memory 136 or external memory 138) that is readable by a machine (e.g., the electronic device 101 or 301). For example, a processor (e.g., the processor 520) of the machine (e.g., the electronic device 301) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to an embodiment of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to an embodiment, each component (e.g., a module or a program) of the above-described components may include a single entity or multiple entities, and some of the multiple entities may be separately disposed in different components. According to an embodiment, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to an embodiment, operations performed by the module, the program, or another component may be carried out sequentially, in parallel, repeatedly, or heuristically, or one or more of the operations may be executed in a different order or omitted, or one or more other operations may be added.

What is claimed is:

1. An electronic device comprising:
an input module;
an output module; and
at least one processor connected to the input module and the output module; and
memory storing instructions that, when executed by the at least one processor individually or collectively, cause the electronic device to:
in case that a user utterance related to a date and time acquired through the input module is identified as a user utterance for performing a function, based on a date and time when the user utterance is acquired, identify whether the date and time detected in the user utterance are interpreted as a plurality of dates and times;
in case that the date and time detected in the user utterance are interpreted as the plurality of dates and times, change the date detected in the user utterance if the date and time when the user utterance is acquired satisfy a first condition for changing the date, and change the time detected in the user utterance if user's activity record information satisfies a second condition for changing the time; and
recommend, through the output module, to perform the function on the changed date or time.

2. The electronic device of claim 1, wherein the instructions, when executed by the at least one processor individually or collectively, cause the electronic device to:
identify a user's sleep time, based on the user's activity record information and, in case that a difference between a sleep end time and a first reference time is smaller than a difference between a sleep start time and the first reference time, activate a function of changing the date and time; and
in case that the function of changing the date and time is activated, identify whether the date and time detected in the user utterance are interpreted as the plurality of dates and times.

3. The electronic device of claim 2,
wherein the instructions, when executed by the at least one processor individually or collectively, cause the electronic device to:
in case that the date and time when the user utterance is acquired are included in a range of the first reference time and a second reference time in a state where the date and time when the user utterance is acquired are identified as a time past the first reference time, identify that the first condition for changing the date is satisfied.

4. The electronic device of claim 3,
wherein the instructions, when executed by the at least one processor individually or collectively, cause the electronic device to:
set the first reference time as midnight; and
set the second reference time as a time after a predetermined time from the midnight or as the sleep start time.

5. The electronic device of claim 1, wherein the instructions, when executed by the at least one processor individually or collectively, cause the electronic device to:
in case that it is identified that the time detected in the user utterance is included in the user's sleep time and is not included in a user's activity time, based on the user's activity record information, identify that the second condition for changing the time is satisfied.

6. The electronic device of claim 5, wherein the instructions, when executed by the at least one processor individually or collectively, cause the electronic device to:
store at least one of sleep time information, alarm setting information, or scheduling information as the user's activity record information.

7. The electronic device of claim 1, wherein the instructions, when executed by the at least one processor individually or collectively, cause the electronic device to:
in case that the date and time when the user utterance is acquired satisfy the first condition for changing the date, change the date detected in the user utterance to a date one day before.

8. The electronic device of claim 1, wherein the instructions, when executed by the at least one processor individually or collectively, cause the electronic device to:
in case that the user's activity record information satisfies the second condition for changing the time, change the time to a time obtained by adding 12 hours to the time detected in the user utterance.

9. The electronic device of claim 1, wherein the instructions, when executed by the at least one processor individually or collectively, cause the electronic device to:
output, through the output module, a message recommending to perform the function on the changed date and time.

10. The electronic device of claim 9,
wherein the instructions, when executed by the at least one processor individually or collectively, cause the electronic device to:
output, through the output module, the message recommending to perform the function on the changed date and time and a message recommending to perform the function on the date and time detected in the user utterance.

11. A method of processing a user utterance by an electronic device, the method comprising:
in case that a user utterance related to a date and time acquired through an input module is identified as a user utterance for performing a function, identifying whether the date and time detected in the user utterance are interpreted as a plurality of dates and times, based on a date and time when the user utterance is obtained;
in case that the date and time detected in the user utterance are interpreted as the plurality of dates and times, changing the date detected in the user utterance if the date and time when the user utterance is obtained satisfy a first condition for changing the date, and changing the time detected in the user utterance if user's activity record information satisfies a second condition for changing the time; and
recommending, through an output module, to perform the function on the changed date or time.

12. The method of claim 11, further comprising:
identifying a user's sleep time, based on the user's activity record information and, in case that a difference between a sleep end time and a first reference time is smaller than a difference between a sleep start time and the first reference time, activating a function of changing the date and time; and
in case that the function of changing the date and time is activated, identifying whether the date and time detected in the user utterance are interpreted as the plurality of dates and times.

13. The method of claim 12, further comprising:
in case that the date and time when the user utterance is acquired are included in a range of the first reference time and a second reference time in a state where the date and time when the user utterance is obtained are identified as a time past the first reference time, identifying that the first condition for changing the date is satisfied.

14. The method of claim 13, wherein the first reference time is set as midnight, and the second reference time is set as a time after a predetermined time from the midnight or as the sleep start time.

15. The method of claim 11, further comprising:
in case that it is identified that the time detected in the user utterance is included in the user's sleep time and is not included in a user's activity time, based on the user's activity record information, identifying that the second condition for changing the time is satisfied.

16. The method of claim 15, further comprising:
store at least one of sleep time information, alarm setting information, or scheduling information as the user's activity record information.

17. The method of claim 11, further comprising:
in case that the date and time when the user utterance is acquired satisfy the first condition for changing the date, change the date detected in the user utterance to a date one day before.

18. The method of claim 11, further comprising:
in case that the user's activity record information satisfies the second condition for changing the time, change the time to a time obtained by adding 12 hours to the time detected in the user utterance.

19. The method of claim 11, further comprising:
output, through the output module, a message recommending to perform the function on the changed date and time.

20. The method of claim 11, further comprising:
output, through the output module, the message recommending to perform the function on the changed date and time and a message recommending to perform the function on the date and time detected in the user utterance.
